(19) United States
(12) Patent Application Publication
(10) Pub. No.: US 2023/0061199 A1
(43) Pub. Date: Mar. 2, 2023

(54) Method and Apparatus for Producing Carbon Monoxide

(71) Applicants: Yasuo Tsujino, Uji-shi (JP); Masaru Nakahara, Otsu-shi (JP)

(72) Inventors: Masaru Nakahara, Otsu-shi (JP); Yasuo Tsujino, Uji-shi (JP)

(21) Appl. No.: 17/892,263

(22) Filed: Aug. 22, 2022

(30) Foreign Application Priority Data
Aug. 24, 2021 (JP) 2021-136112

(51) Int. Cl. C01B 32/40; B01J 19/24; C07C 51/347

(52) U.S. Cl. CPC C01B 32/40; B01J 19/24; C07C 51/347; B01J 2219/00029

(57) ABSTRACT

A method for producing carbon monoxide includes decomposing formic acid, in the presence of water, by hydrothermal reaction under conditions with a temperature T of 350° C. or less and a pressure P being equal to or more than the saturated vapor pressure of water at the temperature T.

### CROSS-REFERENCE TO RELATED APPLICATION

This application claims priority to Japanese Patent Application No. 2021-136112, filed Aug. 24, 2021, the disclosure of which is hereby incorporated by reference in its entirety.

### BACKGROUND OF THE INVENTION

### Field of the Invention

The present invention relates to a method and apparatus for producing carbon monoxide. More specifically, the present invention relates to a method and apparatus for producing carbon monoxide by decomposing formic acid under hydrothermal conditions to obtain carbon monoxide.

### Description of Related Art

Conventionally, there is a known method for producing acetic acid in an industrial scale by oxidizing in a liquid phase various hydrocarbons contained in butane or naphtha which is hydrocarbon (for example, Takuya Kumamoto, “Industrial Synthesis of Formic Acid and Its Utilization”, Chemistry and Education, Japan Chemical Society, 2012, Vol. 60, No. 8, pp. 358-361, <URL: https://www.jstage.jst.go.jp/article/kakyoshi/60/8/60_KJ00008195825/_article/-char/ja/>). The aforementioned prior art document describes that formic acid is obtained as a by-product in the production of acetic acid as mentioned above. Further, the aforementioned prior art document describes that formic acid is also obtained as a by-product in the production of pentaerythritol.

### SUMMARY OF THE INVENTION

### Technical Problem

Since there is not enough demand for formic acid equivalent to the production amount of formic acid, some of the produced formic acid is disposed of or the like. However, disposal of compound that has been once produced is undesirable from the economical aspect. Also such disposal of compounds that have been once produced is undesirable from the aspect of increasing the global environmental load.

Meanwhile, carbon monoxide can be used as a starting material for various organic compounds such as methanol. Therefore, if carbon monoxide can be efficiently produced from formic acid, the produced formic acid can be effectively utilized without being disposed of.

However, it is hard to say that sufficient studies have been made on a method and apparatus for efficient production of carbon monoxide from formic acid.

In view of the above problems, it is an object of the present invention to provide a method and apparatus for producing carbon monoxide enabling the efficient production of carbon monoxide from formic acid.

### Solution to Problem

The method for producing carbon monoxide according to the present invention includes: decomposing formic acid, in the presence of water, by hydrothermal reaction under conditions with a temperature T of 350° C. or less and a pressure P being equal to or more than the saturated vapor pressure of water at the temperature T, to obtain carbon monoxide.

The apparatus for producing carbon monoxide according to the present invention includes: a reaction vessel having a housing space for housing water and formic acid thereinside; a heating device for heating the reaction vessel; and a control part that controls a heating temperature of the reaction vessel by the heating device to have the inside of the reaction vessel at a predetermined pressure, in which the reaction vessel is configured to hermetically house the water and the formic acid in the housing space.

### BRIEF DESCRIPTION OF DRAWINGS

The foregoing and other features of the present invention will become apparent from the following description and drawings of an illustrative embodiment of the invention in which:

FIG. 1 is an entire structure of an apparatus for producing carbon monoxide according to an embodiment of the present invention.

FIG. 2 is an NMR spectra as a function of an initial concentration of formic acid, showing a relationship between a formic acid concentration and a production amount of carbon monoxide.

FIG. 3 is a Raman spectra showing a relationship between each of metal materials and the production amount of carbon monoxide.

FIG. 4 is a graph showing a relationship between a housing rate of formic acid aqueous solution and the production amount of carbon monoxide.

### DESCRIPTION OF THE INVENTION

A method for producing carbon monoxide according to an embodiment of the present invention includes: decomposing formic acid, in the presence of water, by hydrothermal reaction under conditions with a temperature T of 350° C. or less and a pressure P being equal to or more than the saturated vapor pressure of water at the temperature T, to obtain carbon monoxide. The temperature T herein means a temperature of a liquid phase when hydrothermal reaction is performed in liquid phase environment, and means a temperature of a supercritical fluid when hydrothermal reaction is performed in supercritical environment. In the following description, “the embodiment of the present invention” will be simply referred to as “this embodiment”.

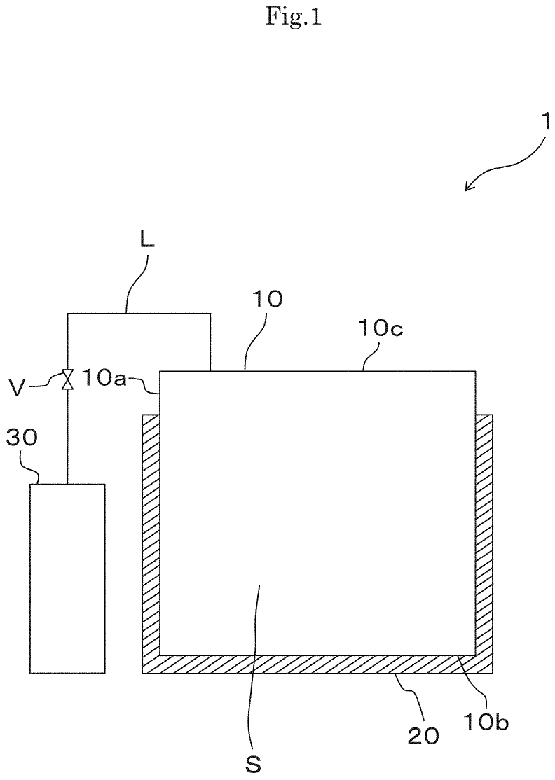

The method for producing carbon monoxide according to this embodiment is performed using a carbon monoxide production apparatus 1, for example, as shown in FIG. 1.

### (Carbon Monoxide Production Apparatus)

The carbon monoxide production apparatus 1 according to this embodiment is a batch reaction apparatus having a housing space S thereinside for housing formic acid (specifically, formic acid aqueous solution including formic acid). Specifically, the carbon monoxide production apparatus 1 according to this embodiment includes a reaction vessel 10 that is formed into a cylindrical shape and has the housing space S for housing formic acid (formic acid aqueous solution), a jacket 20 that covers an outer side surface and a bottom surface of the reaction vessel 10, and a formic acid aqueous solution storage vessel 30 that stores the formic acid aqueous solution including formic acid. The carbon monoxide production apparatus 1 according to this embodiment further includes a pipe L for connecting the reaction vessel 10 with the formic acid aqueous solution storage vessel 30, and a valve V for adjusting an open-close state of the pipe L. The carbon monoxide production apparatus 1 according to this embodiment preferably further includes an inert gas storage vessel (not shown) that stores an inert gas such as nitrogen gas, helium gas, or argon gas, a pipe for connecting the inert gas storage vessel and the reaction vessel 10, and a valve for adjusting an open-close state of the pipe.

The reaction vessel 10 includes a side wall part 10a having a cylindrical shape, a bottom wall part 10b that closes a bottom of the side wall part 10a having a cylindrical shape, and a top wall part 10c that closes a top of the side wall part 10a having a cylindrical shape. In the reaction vessel 10, the bottom wall part 10b and the top wall part 10c close the cylindrical side wall part 10a to bring the housing space S into a sealed space. The formic acid aqueous solution is housed in the housing space S of the reaction vessel 10 from the formic acid aqueous solution storage vessel 30 via the pipe L. The expression “housed in” herein encompasses the narrower concepts such as “poured into”, “loaded in”, and “put into”. The housing of the formic acid aqueous solution into the housing space S can be performed after the pressure in the housing space S is decreased using a decompression pump (not shown) or can be performed in the atmospheric pressure conditions (1.01325×10⁵ Pa (0.101325 MPa)) without decreasing the pressure in the housing space S. From the aspect of easy adjustment of the pressure in the housing space S to the saturated vapor pressure of water or more at the temperature T (i.e., a temperature of 350° C. or less), the housing of the formic acid aqueous solution into the housing space S is preferably performed in the atmospheric pressure conditions without decreasing the pressure in the housing space S. In the reaction vessel 10, at least part of air included in the gas phase in the housing space S can be substituted by the inert gas, or air included in the gas phase in the housing space S can be entirely substituted by the inert gas, by charging the inert gas stored in the inert gas storage vessel into the housing space S after the formic acid aqueous solution is housed in the housing space S. Note that the reaction vessel 10 can include a stirring device (not shown) for stirring the formic acid aqueous solution housed in the housing space S. The reaction vessel 10 including the stirring device enables hydrothermal reaction while the formic acid aqueous solution housed in the housing space S is stirred by the stirring device. This enables more efficient decomposition reaction of formic acid included in the formic acid aqueous solution.

The reaction vessel 10 is a batch vessel as the carbon monoxide production apparatus 1 according to this embodiment is a batch reaction apparatus as described above. The batch vessel herein means a vessel in which the formic acid aqueous solution for use in a single time treatment can be hermetically housed. The reaction vessel 10 has an inner wall surface in contact with the formic acid aqueous solution housed in the reaction vessel 10, and thus the inner wall surface is preferably made of a nonmetallic material. Examples of the material to form the inner wall surface include resin, glass, ceramics, and diamond-like carbon.

The resin can be, for example, plastic such as polyimide (PI), polyamide (PA), polyamide-imide (PAI), polyether sulfone (PES), polyetherimide (PEI), polyether ether ketone (PEEK), aromatic polyester (e.g., PET, PEN), or polyallylene sulfide (PAS), or can be commonly available rubber. In this embodiment, the resin is preferably, for example, a silicone resin, a silicone rubber, a fluororesin, a fluororubber, or an epoxy resin in terms of stability to hydrothermal reaction. Among these resins, the resin is preferably a fluororesin. Examples of the fluororesin include polytetrafluoroethylene (PTFE), tetrafluoroethylene-perfluoro alkyl vinyl ether copolymer (PFA), tetrafluoroethylene-hexafluoro propylene copolymer (FEP), polychlorotrifluoroethylene (PCTFE), tetrafluoroethylene-ethylene copolymer (ETFE), and polyvinylidene fluoride (PVdF). These resins can be employed individually as a forming material of the inner wall surface, or a mixture of two or more of them can be employed as the forming material of the inner wall surface.

Examples of the glass include soda glass, borosilicate glass, quartz glass, and crystal glass.

Examples of the ceramics include alumina (Al₂O₃), zirconia (ZrO₂), titania (TiO₂), silica (SiO₂), silicon carbide (SiC), silicon nitride (Si₃N₄), zircon (ZrO₂.SiO₂), aluminosilicate (Al₂O₃.SiO₂), barium titanate (BaTiO₃), aluminum nitride (AlN), steatite (MgO.SiO₂), forsterite (2MgO.SiO₂), mullite (3Al₂O₃.2SiO₂), and cordierite (2MgO.2Al₂O₃.5SiO₂). These ceramics can be employed individually as a forming material of the inner wall surface, or a mixture of two or more of them can be employed as the forming material of the inner wall surface.

In the reaction vessel 10, the wall that defines the housing space S can be entirely made of the aforementioned materials, or only a surface layer (i.e., a surface layer that forms the inner wall surface of the reaction vessel 10) can be made of the aforementioned materials. The aforementioned materials can be formed into a plurality of layers. For example, the reaction vessel 10 can be configured such that it has a main body made of a metal, and double layers of a glass layer and a resin layer layered on the inner wall surface of the main body.

The forming material of the inner wall surface preferably has an elution amount of metal ion with formic acid at normal temperature (23±2° C.) of 1000 ppm or less. The aforementioned elution amount of metal ion can be measured by, for example, the ICP method.

The inner wall surface of the reaction vessel 10 made of a nonmetallic material as described above can have acid resistance properties. In the case where the inner wall surface is made of a metal material such as stainless steel, the carboxyl group (COOH) included in formic acid (HCOOH) may form an ionic bond with the metal material. However, the inner wall surface made of a nonmetallic material can suppress the formation of the ionic bond as in the above case. That is, it is possible to suppress the formic acid from adhering to the inner wall surface since the formic acid forms the ionic bond with the inner wall surface via the carboxyl group. Thereby, it is possible to more efficiently produce carbon monoxide from the formic acid.

The jacket 20 includes a heating device (not shown) such as a heater. The jacket 20 is configured to heat the reaction vessel 10 with the heating device such as a heater.

The carbon monoxide production apparatus 1 according to this embodiment includes a control part that controls the heating temperature of the reaction vessel 10 by the heating device such as a heater to allow the inside of the reaction vessel 10 to be at a predetermined pressure.

As the formic acid aqueous solution storage vessel 30, any vessel can be used as long as it includes the housing space S for housing the formic acid aqueous solution thereinside. On the other hand, since the formic acid aqueous solution storage vessel 30 has an inner wall surface in contact with the formic acid aqueous solution stored therein, the inner wall surface is preferably made of a nonmetallic material. Examples of a preferred material to form the inner wall surface include resin, glass, ceramics, and diamond-like carbon. The same materials as described above can be used as the resin the glass, and the ceramic.

Note that when the decomposition reaction of formic acid in the housing space S of the reaction vessel 10 proceeds, the formic acid concentration of the formic acid aqueous solution in the housing space S of the reaction vessel 10 is preferably 1.0 mol/L (mole/dm³) or more, more preferably 2.0 mol/L or more.

It has been known that the hydrothermal decomposition reaction (in the absence or presence of catalyst) of formic acid (HCOOH) has a competitive reaction involving two reaction pathways. One is a dehydration step (i.e., the first decomposition reaction) in which formic acid (HCOOH) is decomposed into carbon monoxide (CO) and water (H₂O), and the other is a decarboxylation step (i.e., the second decomposition reaction) in which formic acid (HCOOH) is decomposed into carbon dioxide (CO₂) and hydrogen (H₂). Also, the past findings of the present inventors have revealed that the decomposition reaction rate of formic acid can be represented by formula (1) below when the reaction rate constant of the first decomposition reaction is k₁ and the reaction rate constant of the second decomposition reaction is k₂.

$$\frac{d [ HCOOH ]}{dt} = - {{k}_{1} [ HCOOH ]}^{1.5} - {k}_{2} [ HCOOH ] \qquad ( 1 )$$

In view of formula (1) above, the reaction orders of the first and second decomposition reactions are 1.5, 1, respectively. Therefore it can be found that as the higher concentration of formic acid in the formic acid aqueous solution, the more easy the first decomposition reaction proceeds, dominantly. This finding reveals that it is preferable that the concentration of formic acid be high in the formic acid aqueous solution. On the other hand, the higher the formic acid concentration is, the more the amount of gas components such as carbon monoxide obtained by decomposition of the formic acid is. Accordingly, the pressure in the reaction vessel 10 is raised. When the pressure in the reaction vessel 10 is excessively raised, an exclusive equipment having sufficiently enhanced pressure resistance performance is required to be used as the reaction vessel 10. Therefore, considering that a general equipment is used to perform the method for producing carbon monoxide according to this embodiment, it is preferable that, for example, 3.0 mol/L be employed as the upper limit of the formic acid concentration in the formic acid aqueous solution. Here, in the carbon monoxide production apparatus 1 according to this embodiment, the formic acid aqueous solution supplied from the formic acid aqueous solution storage vessel 30 into the housing space S of the reaction vessel 10 is used, as it is without adjustment of its concentration, for decomposition reaction of formic acid during the supply into the housing space S via the pipe L or after the supply into the housing space S. Therefore, in the carbon monoxide production apparatus 1 according to this embodiment, the formic acid concentration in the formic acid aqueous solution needs to fall within the aforementioned numerical range in the state where the formic acid aqueous solution is housed in the formic acid aqueous solution storage vessel 30.

On the other hand, in the case where the carbon monoxide production apparatus 1 includes, in addition to the formic acid aqueous solution storage vessel 30, a water storage vessel for storing water, the formic acid concentration in the formic acid aqueous solution can be adjusted in the housing space S of the reaction vessel 10 by supplying water from the water storage vessel to the housing space S of the reaction vessel 10. Further, in the case where the pipe L in the carbon monoxide production apparatus 1 is configured to subject water to line mixing, the formic acid concentration in the formic acid aqueous solution can be adjusted in the housing space S of the reaction vessel 10 by subjecting water to line mixing during the supply of the formic acid aqueous solution from the formic acid aqueous solution storage vessel 30 to the housing space S of the reaction vessel 10. Therefore, in such a case, the formic acid concentration in the formic acid aqueous solution can be a value exceeding the aforementioned upper limit in the state where the formic acid aqueous solution is housed in the formic acid aqueous solution storage vessel 30. Moreover, in the case where the reaction vessel 10 is configured to be able to condense the formic acid aqueous solution housed in the housing space S, the formic acid concentration in the formic acid aqueous solution can be a value less than the aforementioned lower limit in the state where the formic acid aqueous solution is housed in the formic acid aqueous solution storage vessel 30.

Note that, as described later, the first decomposition reaction to decompose formic acid into carbon monoxide and water can be easily made to proceed by adding a liquid acid catalyst such as hydrochloric acid or sulfuric acid, which accelerates dehydration reaction, to the formic acid aqueous solution. Therefore, the carbon monoxide production apparatus 1 can include a liquid acid catalyst storage vessel for storing the liquid acid catalyst, a pipe for supplying the liquid acid catalyst stored in the liquid acid catalyst storage vessel into the reaction vessel 10, and a valve for adjusting the opening and closing of the pipe.

As the pipe L, various known pipes made of a metal such as stainless steel can be used. Here, the pipe L serves as a path for supplying the formic acid aqueous solution in the formic acid aqueous solution storage vessel 30 into the housing space S of the reaction vessel 10, and thus the inner wall surface of the pipe L comes into contact with the formic acid aqueous solution when the formic acid aqueous solution is supplied. Therefore, the inner wall surface of the pipe L is also preferably made of a nonmetallic material. Examples of the material to form the inner wall surface include resin, glass, ceramic, and diamond-like carbon. The same materials as described above can be used as the resin, the glass, and the ceramics.

As the valve V, any valves can be used as long as it is attached to a part of the pipe L and it has a function to be able to open and close the pipe conduit.

### Method for Producing Carbon Monoxide

An example of performing the method for producing carbon monoxide according to this embodiment will be described using the carbon monoxide production apparatus 1 which is described above, with reference to FIG. 1.

As described above, the method for producing carbon monoxide according to this embodiment includes decomposing formic acid, in the presence of water, by hydrothermal reaction under conditions with a temperature T of 350° C. or less and a pressure P being equal to or more than the saturated vapor pressure of water at the temperature T, to obtain carbon monoxide.

In the method for producing carbon monoxide according to this embodiment, the decomposing formic acid is performed in the housing space S of the reaction vessel 10. When the decomposing formic acid is performed, first, a predetermined amount of the formic acid aqueous solution housed in the formic acid aqueous solution storage vessel 30 is housed in the housing space S of the reaction vessel 10 via the pipe L. The housing of the formic acid aqueous solution into the housing space S is preferably performed in the atmospheric pressure conditions without decreasing the pressure in the housing space S in terms of easy adjustment of the pressure in the housing space S to be equal to or more than the saturated vapor pressure of water at the temperature T (i.e., a temperature of 350° C. or less). Further, in the case where the carbon monoxide production apparatus 1 according to this embodiment includes the inert gas storage vessel, at least part of the air included in the gas phase in the housing space S can be previously substituted by the inert gas by charging the inert gas stored in the inert gas housing vessel into the housing space S after a predetermined amount of the formic acid aqueous solution is housed in the reaction vessel 10.

Next, the reaction vessel 10 housing a predetermined amount of the formic acid aqueous solution is heated by a heating device such as heater provided in the jacket 20 to have a temperature inside of the reaction vessel 10 (i.e., temperature T) of 350° C. or less. Specifically, the liquid phase (i.e., formic acid aqueous solution) in the housing space S of the reaction vessel 10 is heated by the heating device such as a heater provided in the jacket 20 to have a temperature of 350° C. or less. In the case where hydrothermal reaction is performed at the atmospheric pressure (1.01325×10⁵ Pa (0.101325 MPa)), the temperature of the liquid phase is generally set at a temperature exceeding 100° C. Therefore, the reaction vessel 10 (specifically, the liquid phase in the reaction vessel 10) is preferably heated to a temperature exceeding 100° C. The reaction vessel 10 is heated to a temperature of preferably 200° C. or more, more preferably 250° C. or more. Further, the reaction vessel 10 is preferably heated to a temperature of 300° C. or less.

Here, as described above, it has been known that the hydrothermal decomposition reaction (in the absence or presence of catalyst) of formic acid (HCOOH) includes two reaction pathways. One is a dehydration step (i.e., the first decomposition reaction) in which formic acid (HCOOH) is decomposed into carbon monoxide (CO) and water (H₂O), and the other is a decarboxylation step (i.e., the second decomposition reaction) in which formic acid (HCOOH) is decomposed into carbon dioxide (CO₂) and hydrogen (H₂). The past findings of the present inventors have revealed that the first decomposition reaction is likely to proceed under the kinetic control and that the second decomposition reaction dominantly occurs under the thermodynamic control. Also, the past findings of the present inventors have revealed that the kinetic control has a greater influence at the initial stage of the reaction during the decomposition reaction of formic acid, which allows the first decomposition reaction to easily progress, to thereby enable to easily obtain carbon monoxide. Further, the past findings of the present inventors have revealed that the thermodynamic control increases its influence during the decomposition of formic acid as the evolution of the reaction time, which allows the second decomposition reaction to easily proceed, which results in easily obtaining carbon dioxide that is thermodynamically stable. Therefore, according to the past findings of the present inventors, in order to efficiently obtain carbon monoxide from formic acid, the importance is how to maximize the duration of keeping the influence of the kinetic control in the decomposition reaction of formic acid. That is, the importance is to how to defer the timing of switching from the period during which the first decomposition reaction proceeds to the period during which the second decomposition reaction proceeds in the decomposition reaction of formic acid.

Here, the diligent study of the present inventors has revealed that the higher the temperature in the reaction vessel 10 is, the shorter the time to change into a thermodynamically stable state, that is, the shorter the time of switching from the period during which the first decomposition reaction proceeds to the period during which the second decomposition reaction proceeds. From this finding, it has been revealed that the timing of switching from the period during which the first decomposition reaction proceeds to the period during which the second decomposition reaction proceeds can be relatively deferred by setting the temperature in the housing space S of the reaction vessel 10 to 350° C. or less. Further, the first decomposition reaction produces, as a gas component, only one kind, that is, carbon monoxide. In contrast, the second decomposition reaction produces, as gas components, two kind, that is, carbon dioxide and hydrogen. Thus, when the pressure in the housing space S of the reaction vessel 10 is sufficiently high, the first decomposition reaction proceeds more selectively than the second decomposition reaction according to Le Chatelier's principle. The timing of switching the period during which the first decomposition reaction proceeds to the period during which the second decomposition reaction proceeds is thus relatively deferred to thereby enable to relatively sufficiently proceed the first decomposition reaction. Then, it has been revealed that, in the case where the housing space S of the reaction vessel 10 is a sealed space like the carbon monoxide production apparatus 1 according to this embodiment, the pressure in the housing space S of the reaction vessel 10 can be sufficiently increased by carbon monoxide obtained by the first decomposition reaction, so that the switching from the period during which the first decomposition reaction proceeds to the period during which the second decomposition reaction proceeds can be relatively suppressed. It has also been revealed that when a temperature in the housing space S of the reaction vessel 10 is T, by setting a pressure P in the housing space S of the reaction vessel 10 to be equal to or more than the saturated vapor pressure of water at the temperature T, it is possible to further suppress the switching from the period during which the first decomposition reaction proceeds to the period during which the second decomposition reaction proceeds. That is, the diligent study of the present inventors has revealed that carbon monoxide can be efficiently produced from formic acid by setting the temperature T in the housing space S of the reaction vessel 10 at 350° C. or less and further setting the pressure P in the housing space S of the reaction vessel 10 to be equal to or more than the saturated vapor pressure of water at the temperature T.

The pressure in the housing space S of the reaction vessel 10 is preferably 1.5549 MPa or more at a temperature of 200° C., preferably 3.9776 MPa or more at a temperature of 250° C., preferably 8.5927 MPa or more at a temperature of 300° C., preferably 16.535 MPa or more at a temperature of 350° C. The upper limit of the pressure in the housing space S of the reaction vessel 10 is not particularly limited, but is preferably 50 MPa or less when considering that a general equipment is used as the reaction vessel 10 without using an exclusive equipment having sufficiently enhanced pressure resistance properties.

In terms of easy adjustment of the pressure in the housing space S to be equal to or more than the saturated vapor pressure of water at the temperature T (i.e., a temperature of 350° C. or less) by the gas component obtained by decomposing formic acid included in the formic acid aqueous solution, the formic acid aqueous solution is housed in the housing space S so that, when an entire volume of the housing space S is V_{T}, a gas phase volume Vg based on the entire volume V_{T} of the housing space S is preferably V_{T}/2 or less, more preferably V_{T}/3 or less, still more preferably V_{T}/4 or less. Also, the formic acid aqueous solution can be housed in the housing space S so that the gas phase volume Vg based on the entire volume V_{T} of the housing space S is V_{T}/10 or more. Further, in terms of causing the gas component obtained by the decomposition to be sufficiently present in the housing space S, a formic acid concentration of the formic acid aqueous solution in the housing space S of the reaction vessel 10 is preferably 1.0 mol/L or more, more preferably 2.0 mol/L or more. The upper limit of the formic acid concentration in the formic acid aqueous solution is not particularly limited, but is preferably 3.0 mol/L when considering that a general equipment is used as the reaction vessel 10 without using an exclusive equipment having sufficiently enhanced pressure resistance properties.

When the liquid acid catalyst such as hydrochloric acid or sulfuric acid, which accelerates dehydration reaction, is added to the formic acid aqueous solution, water is easily eliminated from formic acid, which makes it easy to proceed the first decomposition reaction to decompose formic acid into carbon monoxide and water. Therefore, the hydrothermal decomposition reaction of formic acid is preferably performed after the liquid acid catalyst is added to the formic acid aqueous solution. The liquid acid catalyst is included in the formic acid aqueous solution preferably at a concentration of 0.2 mol/L or more, more preferably 0.5 mol/L or more, still more preferably 1 mol/L or more, when the mol concentration of the formic acid is 1 mol/L. The liquid acid catalyst included at a concentration as described above can allow the hydrothermal decomposition reaction of formic acid to further proceed. The upper limit of the concentration of the liquid acid catalyst is not particularly limited. However, in the case where the inner wall surface of the reaction vessel 10 is made of, for example, quartz glass, the concentration of the liquid acid catalyst is preferably 2.0 mol/L or less in terms of sufficiently suppressing occurrence of corrosion or fogging on a surface of the quartz glass. Also in the case where the liquid acid catalyst is not added to the formic acid aqueous solution, an auto-catalytic reaction can be made to easily proceed by formic acid by increasing the formic acid concentration in the formic acid aqueous solution. Thereby, water is easily eliminated from formic acid in the same manner as in the case where the liquid acid catalyst is added to the formic acid aqueous solution, which allows the first decomposition reaction to easily proceed to decompose formic acid into carbon monoxide and water.

In the decomposition reaction of formic acid as described above, the formic acid aqueous solution is not necessarily maintained in a liquid state, but can be in a supercritical fluid. In the case where the decomposition reaction of formic acid as described above has been made to proceed while the formic acid aqueous solution is in a supercritical fluid state, the formic acid aqueous solution can be turned into a liquid state by, for example, cooling the inside of the housing space S of the reaction vessel 10 to about normal temperature (23±2° C.). Then, when the formic acid aqueous solution is turned into a liquid state, carbon monoxide is present in the gas phase in the housing space S of the reaction vessel 10, and thus can be used by, for example, taking the carbon monoxide out from the housing space S of the reaction vessel 10.

The carbon monoxide obtained as described above is used by, for example, being caused to react with hydrogen to obtain methane (CH₄) or methanol (CH₃OH) (see, for example, George A. Olah et al., “Chemical Formation of Methanol and Hydrocarbon (“Organic”) Derivatives from CO₂ and H₂-Carbon Sources for Subsequent Biological Cell Evolution and Life's Origin”, Journal of the American Chemical Society (JACS), year 2017, vol. 139, pages 566-570, and Nobuyuki Matsubayashi and Masaru Nakahara, “Hydrothermal reactions of formaldehyde and formic acid: Free-energy analysis of equilibrium”, The Journal of Chemical Physics (JCP), vol. 122, page 074509 (year 2005)). The reaction to obtain methane or methanol as described above can be performed by supplying hydrogen into the housing space S of the reaction vessel 10, or can be performed in a reaction vessel other than the reaction vessel 10 by taking carbon monoxide out from the housing space S of the reaction vessel 10 and then supplying the carbon monoxide and the hydrogen to the other reaction vessel. Further, regarding the reaction to obtain methanol as described above, in the case where the obtained methanol is partly oxidized and turned into formaldehyde in the housing space S of the reaction vessel 10 or the other reaction vessel, methanol can be again obtained from the formaldehyde by subjecting the formaldehyde to hydrothermal reaction with formic acid in the housing space S of the reaction vessel 10 or the other reaction vessel, in other words, by causing the Cannizzaro reaction to proceed (see, for example, Yasuo Tsujino et al., “Noncatalytic Cannizzaro-type Reaction of Formaldehyde in Hot Water”, Chemistry Letters, year 1999, pages 287-288). In the case where the Cannizzaro reaction is made to proceed in the housing space S of the reaction vessel 10, unreacted formic acid present in the reaction vessel 10 can be used as the formic acid. In the state where the Cannizzaro reaction is made to proceed in the other reaction vessel, formic acid newly supplied in the other reaction vessel can be used as the formic acid.

The method for producing carbon monoxide according to this embodiment can further include generating formic acid by causing carbon dioxide and hydrogen to react with each other. According to this configuration, it is possible to efficiently obtain the formic acid.

The method for producing carbon monoxide according to this embodiment can further include generating hydrogen by electrolyzing water using renewable energy to generate the hydrogen. According to this configuration, it is possible to obtain the hydrogen for use in the generating formic acid, while suppressing the increase of the global environmental load. Examples of the renewable energy include solar power generation, wind power generation, biomass power generation, hydroelectric power generation, and geothermal power generation.

The method for producing carbon monoxide according to this embodiment is preferably performed using a batch reaction apparatus as described above. Specifically, in the method for producing carbon monoxide according to this embodiment, it is preferable that formic acid be housed in a batch vessel (i.e., reaction vessel 10 in this embodiment) to decompose the formic acid in the batch vessel. According to this configuration, it is possible to suppress the decrease of the pressure in the reaction vessel by discharging the gas component(s) obtained by decomposition of the formic acid to the outside during the decomposition of the formic acid. That is, the pressure in the decomposing formic acid can be relatively easily maintained. Thereby, it is possible to more efficiently produce carbon monoxide from formic acid. In the case where a flow-type reaction apparatus is used instead of the batch reaction apparatus, the second decomposition reaction to decompose formic acid into carbon dioxide and hydrogen becomes dominant, or almost only the second decomposition reaction proceeds (see, for example, J. Yu and P. E. Savage, Industrial & Engineering Chemistry Research, vol. 37, page 2 (year 1998), or P. G. Maiella and T. B. Brill, The Journal of Physical Chemistry A (JPCA), vol. 102, page 5886 (year 1998)).

The matters herein disclosed by the present description include the following.

(1)

A method for producing carbon monoxide, the method including: decomposing formic acid, in the presence of water, by hydrothermal reaction under conditions with a temperature T of 350° C. or less and a pressure P being equal to or more than the saturated vapor pressure of water at the temperature T, to obtain carbon monoxide.

According to this configuration, carbon monoxide can be more easily obtained since formic acid is decomposed, in the presence of water, by hydrothermal reaction under conditions with a temperature T of 350° C. or less and a pressure P being equal to or more than the saturated vapor pressure of water at the temperature T. That is, it is possible to efficiently produce carbon monoxide from formic acid.

(2)

The method for producing carbon monoxide as described in (1) above, the method further including: generating formic acid by causing carbon dioxide and hydrogen to react with each other.

According to this configuration, formic acid can be efficiently generated.

(3)

The method for producing carbon monoxide as described in (1) or (2) above, the method further including: generating hydrogen by electrolyzing water using renewable energy.

According to this configuration, it is possible to obtain the hydrogen for use in the generating formic acid, while suppressing the increase of the global environmental load.

(4)

The method for producing carbon monoxide as described in any one of (1) to (3) above, in which the decomposing formic acid further includes: housing the water and the formic acid in a batch vessel; and decomposing the formic acid in the batch vessel.

According to this configuration, the pressure in the decomposing formic acid can be relatively easily maintained since the formic acid is decomposed in the batch vessel. Thereby, it is possible to more efficiently produce carbon monoxide from formic acid.

(5)

The method for producing carbon monoxide as described in (4) above, in which the batch vessel has an inner wall surface in contact with the formic acid housed in the batch vessel, and the inner wall surface is made of a nonmetallic material.

According to this configuration, the adhesion of the formic acid to the inner wall surface of the batch vessel can be relatively suppressed. Thereby, it is possible to more efficiently produce carbon monoxide from formic acid.

(6)

The method for producing carbon monoxide, the method including: decomposing formic acid, in the presence of water, by hydrothermal reaction under conditions with a temperature T of 350° C. or less and a pressure P being equal to or more than the saturated vapor pressure of water at the temperature T, to obtain carbon monoxide, the decomposing formic acid including: housing the water and the formic acid in a batch vessel; and decomposing the formic acid in the batch vessel, in which, in the decomposing formic acid, the water and the formic acid are housed in the batch vessel so that, when an entire volume of the batch vessel is V_{T}, a gas phase volume Vg based on the entire volume V_{T} inside the batch vessel is V_{T}/2 or less.

According to this configuration, it becomes easy to adjust the pressure in the batch vessel to the saturated vapor pressure of water at the temperature T. Thereby, it is possible to more efficiently produce carbon monoxide from formic acid.

(7)

An apparatus for producing carbon monoxide, the apparatus including: a reaction vessel having a housing space for housing water and formic acid thereinside; a heating device for heating the reaction vessel; and a control part that controls a heating temperature of the reaction vessel by the heating device to have the inside of the reaction vessel at a predetermined pressure, in which the reaction vessel is configured to hermetically house the water and the formic acid in the housing space.

According to this configuration, it becomes easy to adjust the inside of the reaction vessel to the conditions, in the presence of water, with a temperature T of 350° C. or less and a pressure P being equal to or more than the saturated vapor pressure of water at the temperature T. Thereby, formic acid can be easily decomposed by hydrothermal reaction, which makes it easier to obtain carbon monoxide. That is, it is possible to efficiently produce carbon monoxide from formic acid.

(8)

The apparatus for producing carbon monoxide as described in (7) above, in which the reaction vessel is a batch vessel having the housing space.

According to this configuration, the pressure in the batch vessel can be relatively easily maintained when formic acid is decomposed by hydrothermal reaction since the formic acid is decomposed inside (i.e., in the housing space of) the batch vessel. Thereby, it is possible to more efficiently produce carbon monoxide from formic acid.

(9)

The apparatus for producing carbon monoxide as described in (8) above, in which the batch vessel has an inner wall surface in contact with the formic acid housed in the batch vessel, and the inner wall surface is made of a nonmetallic material.

According to this configuration, the adhesion of the formic acid to the inner wall surface of the batch vessel can be relatively suppressed. Thereby, it is possible to more efficiently produce carbon monoxide from formic acid.

The method for producing carbon monoxide and the apparatus for producing carbon monoxide according to the present invention are not limited to the configurations of the aforementioned embodiments. Further, various modifications can be made on the method for producing carbon monoxide and the apparatus for producing carbon monoxide according to the present invention without departing from the gist of the present invention.

### EXAMPLES

Hereinafter, the present invention will be described in more detail by way of Examples. Examples in the following are not to limit the scope of the present invention but to provide a more detailed description.

### (Relationship Between a Formic Acid Concentration and a Production Amount of Carbon Monoxide)

Investigation was made on a relationship between a formic acid concentration in a formic acid aqueous solution and a production amount of carbon monoxide. The investigation on the aforementioned relationship was performed in accordance with the following procedures.

(1) A first formic acid aqueous solution having a formic acid concentration of 0.1 M(mol/L), a second formic acid aqueous solution having a formic acid concentration of 0.5 M(mol/L), and a third formic acid aqueous solution having a formic acid concentration of 1.0 M(mol/L) were prepared.
(2) The first formic acid aqueous solution, the second formic acid aqueous solution, and the third formic acid aqueous solution were put into a quartz tube. Each of the first formic acid aqueous solution, the second formic acid aqueous solution, and the third formic acid aqueous solution was poured in the quartz tube individually (i.e., without being mixed with another formic acid aqueous solution). Each of the first formic acid aqueous solution, the second formic acid aqueous solution, and the third formic acid aqueous solution was loaded in the quartz tube so that when an entire volume of the quartz tube is V_{T} and a gas phase volume of the quartz tube is Vg, Vg is V_{T}/2.
(3) The quartz tube was heated until a temperature inside the quartz tube reached 300° C., followed by subjecting each of the formic acid aqueous solutions to decomposition reaction of formic acid for 10 minutes. In the decomposition reaction of formic acid, the pressure in the quartz tube was held at the saturated vapor pressure of water at 300° C.
(4) Each of the formic acid aqueous solutions was subjected to decomposition reaction of formic acid, followed by sampling a gas from a gas phase in the quartz tube, and subjecting the gas to analysis by 13C NMR, to detect the content of carbon monoxide included in the gas phase. The analysis by 13C NMR was performed in the following manner using model type JNM-ECA600 manufactured by JEOL Ltd. as an analyzer.

Analysis by 13C NMR

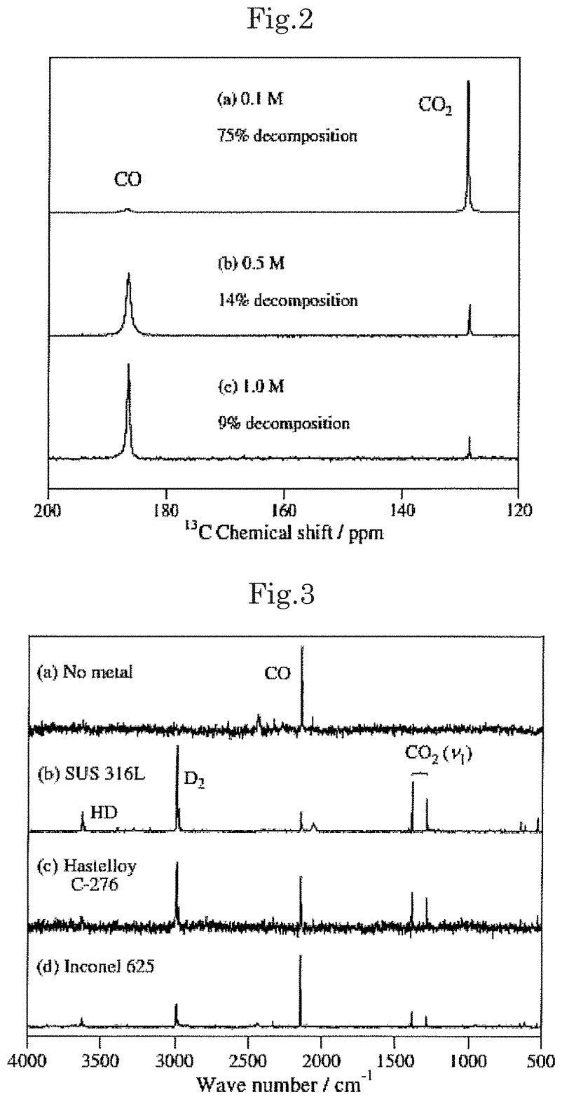

The quartz tube without opening was loaded in an NMR sample tube made of glass having an inner diameter of 5 mm, and set on the analyzer with an observation offset at the gas phase of the quartz tube. Note that, even in the case where the gas phase is located on the lower part and the liquid phase is located on the upper part in the quartz tube during the observation, the gravity did not cause a liquid component in the liquid phase to flow into the lower side (i.e., the gas phase side) due to the influence of the inner pressure and the capillary phenomenon. The analysis temperature was set at 30° C., and the period from a first radio wave irradiation to the next radio wave irradiation was determined by previously measuring a longitudinal relaxation time of a reactant and all the products, and considering the previously measured longitudinal relaxation time (actually, 60 seconds) so as to completely relax a magnetization vector. Double irradiation of proton was not performed since the change of a peak area (i.e., peak height) of carbon atom(s) bonded with a proton caused by NOE (i.e., Nuclear Overhauser effect) influences on quantitativeness. Without locking by a deuterated organic compound, another sample of a 5 mm-NMR tube previously filled with a deuterated organic compound was used for sim adjustment to determine micro magnetic field information, and observed using the determined micro magnetic field information. Further, the quartz tube without opening was quantified using an external reference. The cumulative number was 128, and the number of points was about 32,000. In order to prevent burst of the quartz tube, observation was performed without rotating the sample in a probe. Considering the gas component in the gas phase dissolved in the liquid phase, observation on the liquid phase was also performed in the same manner as in the gas phase as described above. After the aforementioned observations on the gas phase and the liquid phase, the obtained FID (i.e., time variation of the observed magnetization vector signal, Free Induction Decay) was subjected to Fourier transformation to be expressed with a frequency, to determine an area ratio of each peak. In order to improve the accuracy of quantification, the frequency of broadening factor (i.e., window function) was 0.5 Hz or less. Baseline correction on the obtained spectrum was not made. An S/N ratio was 5 or more even at a minimum peak height. The results are shown in FIG. 2.

It can be understood from FIG. 2 that the production amount of carbon monoxide is larger than the production amount of carbon dioxide as the formic acid concentration in the formic acid aqueous solution is higher. From this finding, it can be understood that, as the formic acid concentration in the formic acid aqueous solution is higher, the first decomposition reaction to decompose formic acid into carbon monoxide and water is more likely to proceed than the second decomposition reaction to decompose formic acid into carbon dioxide and hydrogen. It can be also understood that carbon monoxide is selectively generated particularly in the case where the formic acid concentration in the formic acid aqueous solution is 1.0 M.

### (Relationship Between Various Metal Materials and a Production Amount of Carbon Monoxide)

Investigation was made on a relationship between various metal materials and a production amount of carbon monoxide. The investigation on the aforementioned relationship was performed in accordance with the following procedures.

(1) A formic acid deuterated oxide solution having a formic acid concentration of 2 M(mol/L) was prepared.
(2) A quartz tube pouring only the formic acid deuterium oxide solution was prepared as Test Body No. 1, a quartz tube putting the formic acid deuterated oxide solution and SUS powder was prepared as Test Body No. 2, a quartz tube loading the formic acid deuterated oxide solution and Hastelloy powder was prepared as Test Body No. 3, and a quartz tube enclosing the formic acid deuterated solution and Inconel powder was prepared as Test Body No. 4. The SUS powder was produced by milling SUS 316L, the Hastelloy powder was produced by milling Hastelloy C-276, and the Inconel powder was produced by milling Inconel 625. Each of Test Body No. 1 to Test Body No. 4 houses the formic acid deuterated oxide solution, or the formic acid deuterated oxide solution along with any one of various powders so that when an entire volume of the quartz tube is V_{T} and a gas phase volume of the quartz tube is Vg, Vg is V_{T}/2.
(3) Each of Test Body No. 1 to Test Body No. 4 was heated until a temperature inside the quartz tube reached 250° C., followed by subjecting each of the formic acid deuterated oxide solutions to decomposition reaction of formic acid for 1 hour. In the decomposition reaction of formic acid, the pressure in the quartz tube was held at the saturated vapor pressure of water at 250° C.
(4) Each of the formic acid deuterated oxide solutions was subjected to decomposition reaction of formic acid, followed by sampling a gas from a gas phase in the quartz tube, and subjecting the gas to analysis by Raman spectroscopy, to detect the content of carbon monoxide included in the gas phase. The analysis by Raman spectroscopy was performed in the following manner using model type RM1000B manufactured by Renishaw plc. as an analyzer.

Analysis by Raman Spectroscopy

The quartz tube without opening was set on the analyzer with an observation offset at the gas phase of the quartz tube. The measured temperature was 30° C. The setting of the quartz tube was made in accordance with the setting described in the aforementioned NMR observation. The results are shown in FIG. 3.

It can be understood from FIG. 3 that carbon monoxide is selectively generated in Test Body No. 1 that houses only the formic acid deuterated oxide solution in the quartz tube. In contrast, it can be understood that carbon dioxide and deuterium are generated as well as carbon monoxide in Test Body No. 2 to Test Body No. 4 each pouring the formic acid deuterated oxide solution and any one of various metal powders (i.e., SUS powder, Hastelloy powder, and Inconel powder) in the quartz tube. That is, it can be understood that the second decomposition reaction to decompose formic acid into carbon dioxide and hydrogen is likely to proceed in the case where the inner wall surface of the reaction vessel 10 is made of a metal in the carbon monoxide production apparatus 1 as shown in FIG. 1. This fact reveals that the inner wall surface of the reaction vessel 10 made of a nonmetallic material enables to selectively allow the first decomposition reaction to proceed to decompose formic acid into carbon monoxide and water, while suppressing the progress of the second decomposition reaction.

### (Relationship Between a Housing Rate of the Formic Acid Aqueous Solution and a Production Amount of Carbon Monoxide)

Investigation was made on a relationship between a filling rate of the formic acid aqueous solution in the reaction vessel and a production amount of carbon monoxide. The investigation on the aforementioned relationship was performed in accordance with the following procedures.

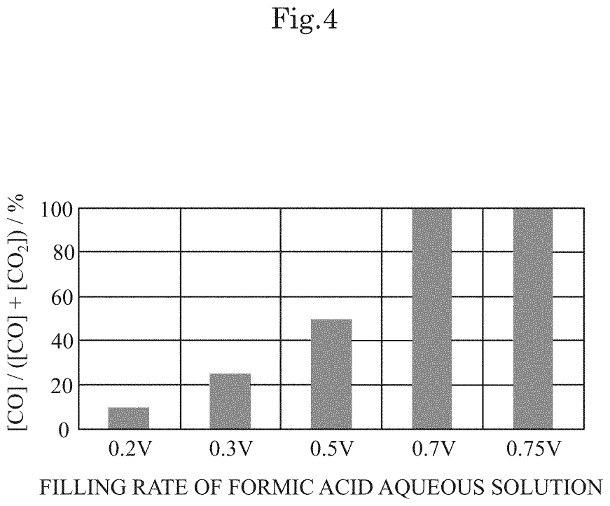

(1) A formic acid aqueous solution having a formic acid concentration of 2 M(mol/L) was prepared.
(2) A quartz tube pouring the formic acid aqueous solution so that Vg is 0.8 V_{T} (i.e., the filling rate of the formic acid aqueous solution is 0.2 V_{T}) when an entire volume of the quartz tube is V_{T} and a gas phase volume of the quartz tube is Vg was prepared as Test Body No. 1′, a quartz tube putting the formic acid aqueous solution so that Vg is 0.7 V_{T} (i.e., the filling rate of the formic acid aqueous solution is 0.3 V_{T}) was prepared as Test Body No. 2′, a quartz tube loading the formic acid aqueous solution so that Vg is 0.5 V_{T} (i.e., the filling rate of the formic acid aqueous solution is 0.5 V_{T}) was prepared as Test Body No. 3′, a quartz tube enclosing the formic acid aqueous solution so that Vg is 0.3 V_{T} (i.e., the filling rate of the formic acid aqueous solution is 0.7 V_{T}) was prepared as Test Body No. 4′, and a quartz tube encapsulating the formic acid aqueous solution so that Vg is 0.25 V_{T} (i.e., the filling rate of the formic acid aqueous solution is 0.75 V_{T}) was prepared as Test Body No. 5′.
(3) Each of Test Body No. 1′ to Test Body No. 5′ was heated until a temperature inside the quartz tube reached 250° C., followed by subjecting each of the formic acid aqueous solutions to decomposition reaction of formic acid for 12 hours. In the decomposition reaction of formic acid, the pressure in the quartz tube was held at the saturated vapor pressure of water at 250° C.
(4) Each of the formic acid aqueous solutions was subjected to decomposition reaction of formic acid, followed by sampling a gas from a gas phase in the quartz tube, and subjecting the gas to analysis by 13C NMR, to quantify the concentration of carbon monoxide and the concentration of carbon dioxide included in the gas phase. Then, the percentage of the carbon monoxide to the sum of the concentration of carbon monoxide and the concentration of carbon dioxide was calculated. The analysis by 13C NMR was performed in the same manner as described in the section for (Relationship between a formic acid concentration and a production amount of carbon monoxide). The results are shown in FIG. 4.

It can be understood from FIG. 4 that the production amount of carbon monoxide increases as the housing rate of the formic acid aqueous solution is higher, in other words, as the occupation rate of gas phase volume Vg is smaller. In particular, it can be understood that the percentage of the concentration of carbon monoxide relative to the sum of the concentration of carbon monoxide and the concentration of carbon dioxide is 100%, that is, only carbon monoxide is selectively generated, when the housing rate of formic acid is 0.7 V or more (in other words, when the occupation rate of gas phase volume Vg is 0.3 V or less).

Although the method for producing carbon monoxide and the apparatus for producing carbon monoxide according to the present invention are as described above, the present invention is not limited to the aforementioned embodiment and the design may be appropriately changed within the scope where the present invention is intended. Also, the functional effect of the present invention is not limited to the aforementioned embodiment. That is, the embodiments disclosed herein should be assumed as not limitations but exemplifications in all aspects. The scope of the present invention is described not by the above description but by the claims. Further, the scope of the present invention is intended to include the scope equivalent to the claims and all the changes in the claims.

What is claimed is:

1. A method for producing carbon monoxide, the method comprising: decomposing formic acid, in the presence of water, by hydrothermal reaction under conditions with a temperature T of 350° C. or less and a pressure P being equal to or more than the saturated vapor pressure of water at the temperature T, to obtain carbon monoxide.

2. The method for producing carbon monoxide according to claim 1, the method further comprising: generating formic acid by causing carbon dioxide and hydrogen to react with each other.

3. The method for producing carbon monoxide according to claim 2, the method further comprising: generating hydrogen by electrolyzing water using renewable energy.

4. The method for producing carbon monoxide according to claim 1, wherein the decomposing formic acid further comprises: housing the water and the formic acid in a batch vessel; and decomposing the formic acid in the batch vessel.

5. The method for producing carbon monoxide according to claim 4, wherein the batch vessel has an inner wall surface in contact with the formic acid housed in the batch vessel, and the inner wall surface is made of a nonmetallic material.

6. A method for producing carbon monoxide, the method comprising: decomposing formic acid, in the presence of water, by hydrothermal reaction under conditions with a temperature T of 350° C. or less and a pressure P being equal to or more than the saturated vapor pressure of water at the temperature T, to obtain carbon monoxide, the decomposing formic acid comprising: housing the water and the formic acid in a batch vessel; and decomposing the formic acid in the batch vessel, wherein, in the decomposing formic acid, the water and the formic acid are housed in the batch vessel so that, when an entire volume of the batch vessel is V_{T}, a gas phase volume Vg based on the entire volume V_{T} inside the batch vessel is V_{T}/2 or less.

7. An apparatus for producing carbon monoxide, the apparatus comprising: a reaction vessel having a housing space for housing water and formic acid thereinside; a heating device for heating the reaction vessel; and a control part that controls a heating temperature of the reaction vessel by the heating device to have the inside of the reaction vessel at a predetermined pressure, in which the reaction vessel is configured to hermetically house the water and the formic acid in the housing space.

8. The apparatus for producing carbon monoxide according to claim 7, wherein the reaction vessel is a batch vessel having the housing space.

9. The apparatus for producing carbon monoxide according to claim 8, wherein the batch vessel has an inner wall surface in contact with the formic acid housed in the batch vessel, and the inner wall surface is made of a nonmetallic material.
